(19) United States
(12) Patent Application Publication
(10) Pub. No.: US 2012/0122190 A1
(43) Pub. Date: May 17, 2012

(54) Red Mold Mutant that can Increase the Production of Monascus Metabolites

(76) Inventors: HSIA-CHING HSIAO, Taipei City (TW); TZU-MING PAN, Sijhih City (TW)

(21) Appl. No.: 12/946,823

(22) Filed: Nov. 15, 2010

(51) Int. Cl. C12N 1/14

(52) U.S. Cl. 435/254.1

(57) ABSTRACT

The present invention discloses a Red mold mutant that can increase the production of monascus metabolites, wherein the Red mold mutant is Monascus purpureus NTU 568 and deposited in Agricultural Research Culture Collection (NRRL) in Nov. 13, 2009, and the accession number is NRRL 50338. The disclosed Monascus purpureus NTU 568 can increase the production amount of monascin and ankaflavin.

### BACKGROUND OF THE INVENTION

1. Field of the Invention

The present invention relates to a Red mold (Monascus purpureus) mutant, and more particularly to a Red mold mutant which can increase the production of monascus metabolites.

2. Description of the Prior Art

Red mold is a traditional fermentable fungus in China, and it has been applied in food products for thousands of years. The specific functions and applications of Red mold are recorded on ancient books of China. In addition of being helpful to the blood circulation and lipid metabolism of human body, Red mold can also be used as a food dye and a spice. Monascus pigment is an excellent and safe natural red pigment, which can make food to present red color.

Red mold has been proven to produce many functional secondary metabolites in previous studies, such as monacolin K. Monacolin K is also referred to as lovastatin, and its major function is being a cholesterol synthesis inhibitor. The primary mechanism of monacolin K is to specifically inhibit the activity of the enzyme HMG-CoA reductase (3-hydroxy-3-methylglutaryl coenzyme A reductase), which is required in a cholesterol synthesis rate-determining step. When the activity of the HMG-CoA reductase enzyme is inhibited, mevalonic acid cannot be produced. Consequently, the synthesis of cholesterol is inhibited, therefore achieves the effect of lowering cholesterol.

Because monacolin K can inhibit the metabolism of isoprene, monacolin K is also a useful tool for the researches of the composition of cell membrane, the hormone of an insect, the regulation in plant growth, animal cell differentiation, and chromosome replication.

Although most species of the Red mold can produce monacolin K, the amount of production is generally not high enough. Thus, conventional species of Red mold can not bring the function of monacolin K into full play.

Moreover, the secondary metabolites of Red mold further include monascin and ankaflavin, which are anti-inflammatory substances. In recent years, many researches prove that these two substances further have the effect of preventing atherosclerosis. However, the contents of monascin and ankaflavin in most Monascus species are not high enough to treat inflammation and prevent atherosclerosis, thus the effects of above functions are limited.

In view of this, it is necessary to provide a novel species of Red mold that can increase the production amount of monascin and ankaflavin, so as to elevate the efficiency of related functions of Red mold.

### SUMMARY OF THE INVENTION

In view of the above shortcomings of the prior art, the inventor of the present invention resorted to past experience, imagination, and creativity, performed experiments and researches repeatedly, and eventually devised the present invention, a Red mold mutant can increase the production of monascus metabolite.

The major objective of the present invention is to provide the Red mold mutant can increase the production of monascus metabolites, which can increase the production amount of monascin and ankaflavin, so as to elevate the efficiency of related functions of Red mold.

According to the above objective, the present invention provides a Red mold mutant can increase the production of monascus metabolites, wherein the Red mold mutant is Monascus purpureus NTU 568 and deposited in Agricultural Research Culture Collection (NRRL) in Nov. 13, 2009, and the accession number being NRRL 50338.

### BRIEF DESCRIPTION OF THE DRAWINGS

For a complete understanding of the aspects, structures and techniques of the invention, reference should be made to the following detailed description and accompanying drawings wherein:

FIG. 1 is a trend graph of the production of monacolin K and the pH value in a liquid culture when the fermentation substrates are rice and dioscorea respectively; and

FIG. 2 is a trend graph of the production of monacolin K and citrinin in red mold dioscorea under different pH values.

### DETAILED DESCRIPTION OF THE PREFERRED EMBODIMENTS

To achieve the foregoing objectives and effects, the inventors screen the Red mold mutant in the mutants of Monascus purpureus, thus achieving a Red mold mutant disclosed in the present invention which can increase the production of monascus metabolites.

The Red mold mutant provided by the present invention is Monascus purpureus NTU 568 and deposited in Agricultural Research Culture Collection (NRRL) in Nov. 13, 2009, and the given accession number is NRRL 50338.

The characteristics of Monascus purpureus NTU 568 includes rapid growth, strong ability of starch hydrolysis, and strong ability of metabolites production. The basic culture manner of Monascus purpureus NTU 568 is to be cultured with a medium containing 2% of rice powder, the best culture temperature is 30° C., the best culture time is 48 hours, the best culture pressure is 1 atm, and the growth of Monascus purpureus NTU 568 is oxygen dependent.

The method for testing the viability of Monascus purpureus NTU 568 is the following: after transferring Monascus purpureus NTU 568 from a slant tube to a potato dextrose agar (PDA) and culturing it for 5 days, digging 3 blocks of hyphal body with volume of 1 cm³; inoculating the hyphal body into a 100 ml medium containing 2% of rice powder; and culturing the Monascus purpureus NTU 568 with shaking manner under 30° C. and 200 rpm for 48 hours. If the cultured liquid presents red color after 48 hours of culture, the viability of Monascus purpureus NTU 568 is well.

The storage method for Monascus purpureus NTU 568 is to store on a PDA medium in a slant tube under 4° C., and it has to be sub-cultured every 3 months.

Monascus purpureus NTU 568 of the present invention can increase the production of monacolin K, that is, the production amount of monacolin K is higher in Monascus purpureus NTU 568 than in other species of Monascus purpureus. In order to make the production amount of monacolin K reach a maximum level, Monascus purpureus NTU 568 can be cultured with a specific culture medium under a specific culture condition. The specific culture medium contains a substrate in a ratio of 5%, and the substrate can be rice powder, dioscorea powder, or any material containing starch. In the above culture condition, a culture temperature is 30° C., an amount of ventilation is 4 vvm, a rotational speed is 200 rpm, and a culture time is 12 days.

In order to prove that Monascus purpureus NTU 568 of the present invention has the effect of increasing the production amount of monacolin K, the following analyzes and compares the function of Monascus purpureus NTU 568 with several experiments.

Referring to table 1, it illustrates the production amount of monacolin K of several species of Monascus purpureus cultured with liquid manner and fermented with rice and dioscorea respectively. The results demonstrate that regardless of the fermented substrate is rice or dioscorea, the production amount of monacolin K in Monascus purpureus NTU 568 is higher than other 6 species of Monascus purpureus. It can be seen that Monascus purpureus NTU 568 of the present invention indeed can increase the production amount of monacolin K. Furthermore, the production amount of monacolin K in Monascus purpureus NTU 568 fermented with dioscorea is higher than that with rice, thus, dioscorea is more suitable than rice to be the fermented substrate for producing monacolin K.

TABLE 1

| species of Monascus | | Production amount of |
|---|---|---|
| purpureus | Fermented substrate | monacolin K (mg/g) |
| M. sp. CA 505 | rice | 2.42 |
| | dioscorea | 8.45 |
| M. purpureus NTU 568 | rice | 7.62 |
| | dioscorea | 18.92 |
| M. sp. CH 001 | rice | 5.92 |
| | dioscorea | 17.65 |
| M. purpureus NTU 601 | rice | 0.58 |
| | dioscorea | 3.54 |
| M. purpureus NTU 301 | rice | 0.51 |
| | dioscorea | 3.08 |
| M. anka M13 | rice | 0.11 |
| | dioscorea | 0.63 |
| M. sp. KT | rice | 0.36 |
| | dioscorea | 3.89 |

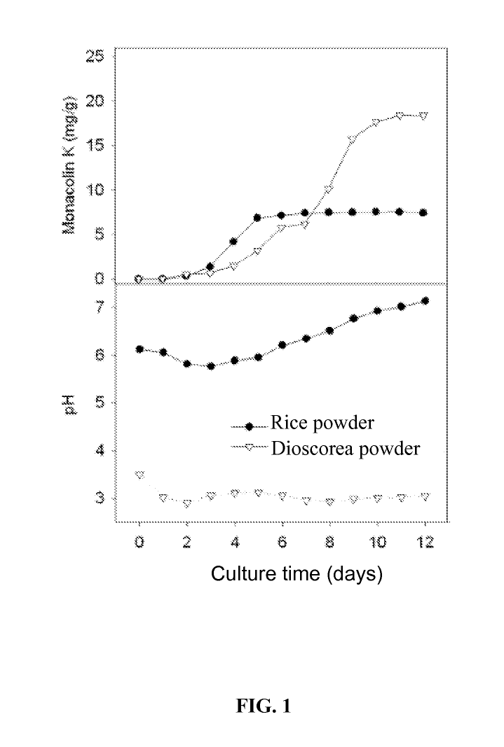

Referring to FIG. 1, which is a trend graph of the production of monacolin K and the pH value in a liquid culture when the fermentation substrates are rice and dioscorea respectively. In FIG. 1, the upper part presents the concentration of monacolin K (mg/g), and the lower part presents the pH value; the horizontal axis presents the culture time (days); the black dots present that the substrate is rice powder (5%); the white triangles present that the substrate is dioscorea powder (5%). The results demonstrate that when the substrate is rice powder, the variation range of the pH value is from 5.5 to 7.1, and the production of monacolin K is stop after 5 days. When the substrate is dioscorea, the starting pH value is 3.5, and with the lapse of time, the pH value is decreased to 3.0, and then the pH value in the following culture time has no much change; regarding to monacolin K, the production amount of monacolin K increases with time, and stop to increase after 11 days. Thus, pH value is an important factor affecting the production of monacolin K.

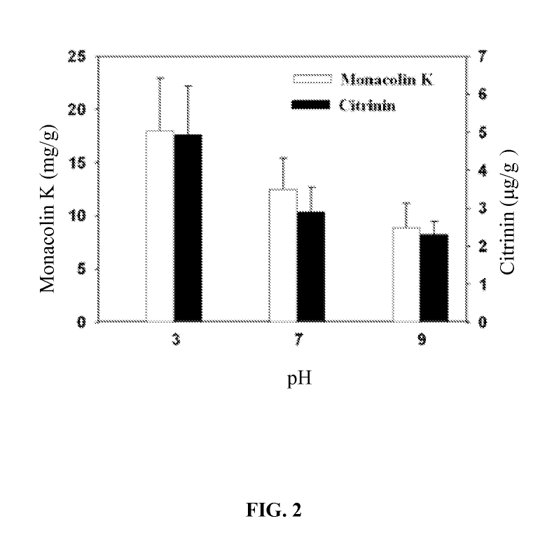

Referring to FIG. 2, which is a trend graph of the production of monacolin K and citrinin in red mold dioscorea under different pH values. In FIG. 2, white column presents the concentration of monacolin K (mg/g); black column presents the concentration of citrinin (μg/g). The results demonstrate that under different pH values, the content ratios of monacolin K and citrinin are approximately similar, but the production amount of monacolin K is higher in an acidic environment than in a neutral environment or an alkaline environment.

Furthermore, Monascus purpureus NTU 568 of the present invention can elevate the production amount of monascin and ankaflavin. In order to prove the effect, the present invention analyze Monascus purpureus NTU 568 and other 11 kinds of Red mold species/Red mold products sold on the market at the same time for comparing the production amount of monascin and ankaflavin. The experiments utilize Thin-Layer Chromatography (TLC) and High Performance Liquid Chromatography (HPLC) for analyzing. Referring to the following table 2, which illustrates the production amount of Monascus purpureus NTU 568 and other 11 kinds of Red mold species/Red mold products sold on the market. The results demonstrate that the 11 kinds of Red mold species/Red mold products sold on the market are not to be detected the content of monascin and ankaflavin, and the production amount of monascin in Monascus purpureus NTU 568 of the present invention is 11.65 mg/0.5 g and the production amount of ankaflavin is 1.77 mg/0.5 g. It will be seen from this that Monascus purpureus NTU 568 of the present invention can increase the production amount of monascin and ankaflavin related to other species of Red mold, so as to elevate the effect of anti-inflammation and atherosclerosis prevention.

TABLE 2

| Red mold species/ | Production amount of | Production amount of |
|---|---|---|
| products | monascin (mg/0.5 g) | ankaflavin (mg/0.5 g) |
| Monascus product | — | — |
| (NU SKIN) | | |
| Monascus product (CPC) | — | — |
| Monascus product 1 | — | — |
| (jensheng) | | |
| Monascus product | — | — |
| (Koda) | | |
| Monascus product | — | — |
| (Prince Pharmaceutical) | | |
| Monascus product | — | — |
| (Standard) | | |
| Monascus product 2 | — | — |
| (jensheng) | | |
| Monascus product 3 | — | — |
| (jensheng) | | |
| Monascus product | — | — |
| (SANHE) | | |
| Monascus product | — | — |
| (YU-SHAN) | | |
| Monascus product | — | — |
| (WEI-CHUAN) | | |
| Monascus purpureus | 11.65 | 1.77 |
| NTU 568 | | |

By the detailed description of the overall structure and technical content of the present invention, the following advantages of the present invention can be derived:
1. Monascus purpureus NTU 568 of the present invention can produce much more amount of monacolin K relative to other species of Monascus purpureus, so that Monascus purpureus NTU 568 can elevate the effect of related function of monacolin K.
2. Monascus purpureus NTU 568 of the present invention can increase the production amount of monascin and ankaflavin related to other species of Red mold, so as to elevate the effect of anti-inflammation and atherosclerosis prevention.

It should be understood that the embodiments of the present invention described herein are merely illustrative of the technical concepts and features of the present invention and are not meant to limit the scope of the invention. Those skilled in the art, after reading the present disclosure, will know how to practice the invention. Various variations or modifications can be made without departing from the spirit of the invention. All such equivalent variations and modifications are intended to be included within the scope of the invention.

As a result of continued thinking about the invention and modifications, the inventors finally work out the designs of the present invention that has many advantages as described above. The present invention meets the requirements for an invention patent, and the application for a patent is duly filed accordingly. It is expected that the invention could be examined at an early date and granted so as to protect the rights of the inventors.

What is claimed is:

1. A Red mold mutant that can increase the production of monascus metabolites, wherein the Red mold mutant is Monascus purpureus NTU 568 which was deposited in Agricultural Research Culture Collection (NRRL) in Nov. 13, 2009, and the accession number of Monascus purpureus NTU 568 being NRRL 50338.

2. The Red mold mutant according to claim 1, wherein the Monascus purpureus NTU 568 can increase the productions of monascin and ankaflavin.

3. The Red mold mutant according to claim 2, wherein the Monascus purpureus NTU 568 is cultured with a specific culture medium under a specific culture condition.

4. The Red mold mutant according to claim 3, wherein the specific culture medium contains a substrate in a percentage of 2%.

5. The Red mold mutant according to claim 4, wherein the substrate is selected from a group consisting rice powder and dioscorea powder.

6. The Red mold mutant according to claim 3, wherein the specific culture condition comprises a culture temperature being 30° C.; a culture time being 48 hours; a culture pressure being 1 atm (1 atmosphere pressure); and requirement for oxygen.
